(19) United States
(12) Patent Application Publication
(10) Pub. No.: US 2011/0172274 A1
(43) Pub. Date: Jul. 14, 2011

(54) FLUORO-SUBSTITUTED 3,4-DIARYL-4,5-DIHYDRO-1H-PYRAZOLE-1-CARBOXAMIDINE DERIVATIVES HAVING CB1-ANTAGONISTIC ACTIVITY

(76) Inventors: Josephus H.M. Lange, Weesp (NL); Bernard J. Vliet, Van, Weesp (NL)

(21) Appl. No.: 12/999,494

(22) PCT Filed: Jun. 15, 2009
(86) PCT No.: PCT/EP2009/057323

§ 371 Date: Mar. 15, 2011

Related U.S. Application Data
(60) Provisional application No. 61/061,716, filed on Jun. 16, 2008.

(30) Foreign Application Priority Data
Jun. 16, 2008 (EP) 08158309.8

(51) Int. Cl. A61K 31/454; C07D 401/12; A61P 3/04; A61P 9/00; A61P 25/30; A61P 1/16; A61P 31/00

(52) U.S. Cl. 514/326; 546/211

(57) ABSTRACT

This invention concerns fluorinated 3,4-diaryl-4,5-dihydro-1H-pyrazole-1-carboxamidine derivatives as cannabinoid-CB₁ receptor antagonists, methods for preparing these compounds, novel intermediates useful for the synthesis of said compounds, methods for the preparation of these intermediates, pharmaceutical compositions containing one or more of these dihydropyrazole derivatives as active ingredient, as well as the use of these pharmaceutical compositions for the treatment of obesity and obesity-related cardiovascular disorders, drug addiction, cognition deficits, liver fibrosis and inflammatory disorders. The compounds have the general formula (I)

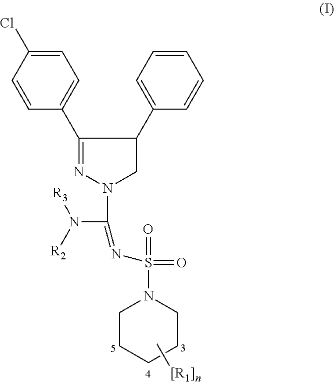

wherein the symbols have the meanings given in the specification.

### TECHNICAL FIELD

This invention relates to pharmaceutical and organic chemistry, and provides fluoro-substituted 3,4-diaryl-4,5-dihydro-1H-pyrazole-1-carboxamidine derivatives, intermediates, formulations and methods.

### BACKGROUND

Cannabinoid (CB) receptors are part of the endocannabinoid system, involved in neurological-, psychiatric-, cardiovascular-, gastrointestinal-, reproductive- and eating disorders, as well as in cancer (De Petrocellis, 2004; Di Marzo, 2004, 2008; Lambert, 2005; Vandevoorde, 2005).

CB₁ receptor antagonists from different structural classes have been described (Lange, 2004, 2005^{B}; Barth, 2005; Muccioli, 2006; Hepworth, 2006; Högenauer, 2007). These compounds, as well as CB₁ receptor inverse agonists, are suggested to be of therapeutic value in treating obesity and obesity-related cardiovascular disorders such as diabetes type II and several other disorders, including drug addiction, cognition deficits, liver fibrosis and inflammatory disorders (Colombo, 1998; Cooke, 2006; Van Gaal, 2005; Antel, 2006; Le Foll, 2005; Maldonado, 2006; Castellano, 2003; Wolff, 2003; Teixeira-Clerc, 2006; Sarnataro, 2006; Lambert, 2007; Costa, 2007; Crozi, 2007).

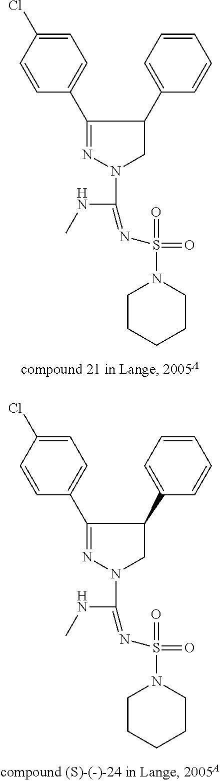

Examples 55 and 100 disclosed in WO 03/026648 as cannabinoid CB₁ receptor antagonists are 3,4-diaryl-4,5-dihydro-1H-pyrazole derivatives structurally related to the compounds of the present invention. The affinity of both compounds (compounds 21 and (S)-(−)-24 in Lange, 2005^{A}) for human CB₁ receptors was found to be 152 and 58 nM respectively.

### DISCLOSURE

This invention relates to a compound of formula (I):

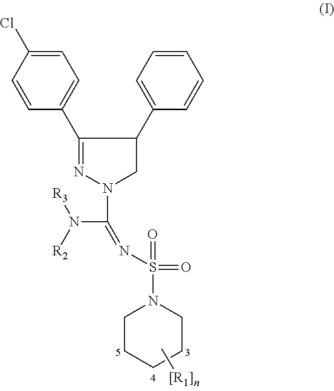

or a tautomer, stereoisomer, N-oxide, or a pharmacologically acceptable salt, of any of the foregoing, wherein:
n is 1 or 2,
when n=1, R₁ is chosen from F or CF₃, at either the 3- or 4-position of the piperidine ring,
when n=2, R₁ is F, either both at the 3- or 4-positions, or one at the 3-position, and the second either at position 4 or position 5,
R₂ is chosen from H or (C₁₋₃)-alkyl,
R₃ is chosen from H or methyl.

These novel fluoro-substituted piperidine derivatives, are potent and selective antagonists or inverse agonists of human cannabinoid-CB₁ receptors, considerably more potent than the corresponding non-fluorinated compounds disclosed in WO 03/026648.

The invention also relates, in some embodiments, to a compound of formula (I) wherein R₃ is H, and the other symbols have the meanings as given above.

Other embodiments provide a compound of formula (I) wherein R₂ is chosen from methyl and ethyl, R₃ is H, and the other symbols have the meanings as given above.

In another embodiment the invention relates to compounds of formula (I) wherein the carbon atom at the 4-position of the 4,5-dihydropyrazole ring has the (S)-configuration according to the Cahn-Ingold-Prelog system.

The compounds of formula (I), as well as tautomers, stereoisomers, N-oxides, and pharmacologically acceptable salts of any of the foregoing, are potent and selective antagonists or inverse agonists of human cannabinoid-CB₁ receptors. They are useful in treating disorders involving cannabinoid neurotransmission, or treatable by manipulating those receptors. For instance in obesity and obesity-related cardiovascular disorders such as diabetes type II, and several other disorders, including drug addiction, cognition deficits, liver fibrosis and inflammatory disorders

Other embodiments of the invention include:
pharmaceutical compositions for treating, for example, a disorder or condition treatable by blocking human cannabinoid-CB₁ receptors, the composition comprising a compound of formula (I), and a pharmaceutically acceptable carrier;
methods of treating a disorder or condition treatable by blocking human cannabinoid-CB₁ receptors, the method comprising administering to a mammal in need of such treating a compound of formula (I);
pharmaceutical compositions for treating, for example, a disorder or condition chosen from obesity and obesity-related cardiovascular disorders, drug addiction, cognition deficits, liver fibrosis and inflammatory disorders;
pharmaceutical compositions for treating a disorder or condition chosen from the disorders listed herein, the compositions comprising a compound of formula (I), and a pharmaceutically acceptable carrier;
methods for treating a disorder or condition chosen from the disorders listed herein, comprising administering to a patient in need of such treating a compound of formula (I).
methods of antagonizing human cannabinoid-CB₁ receptors comprising administering to a subject in need thereof, an pharmaceutically effective amount of a compound of formula (I);

The invention also provides the use of a compound of formula (I) for the manufacture of medicament.

The invention further relates to combination therapies wherein a compound of the invention, or a pharmaceutical composition or formulation comprising a compound of the invention, is administered concurrently or sequentially or as a combined preparation with another therapeutic agent or agents, for treating one or more of the conditions listed. Such other therapeutic agent(s) may be administered prior to, simultaneously with, or following the administration of the compounds of the invention.

The invention also provides compounds, pharmaceutical compositions, kits and methods for treating one or more of the conditions listed, the method comprising administering to a patient in need of such treating a compound of formula (I).

The compounds of the invention are antagonists of human cannabinoid-CB₁ receptors. This activity can be readily demonstrated using one or more of the assays described herein or known in the art.

The invention also provides methods of preparing the compounds of the invention and the intermediates used in those methods.

The compounds and intermediates described herein can, if desired, be isolated and purified by any suitable separation or purification procedure such as, filtration, extraction, crystallization, column chromatography, thin-layer chromatography, thick-layer chromatography, preparative low or high-pressure liquid chromatography, or a combination of these procedures. Illustrations of suitable separation and isolation procedures can be taken from the preparations and examples. However, other equivalent separation or isolation procedures could be used, too.

The compounds of the present invention contain one or more asymmetric centers and can thus occur as racemates and racemic mixtures, single enantiomers, diastereomeric mixtures and individual diastereomers.

Depending on the nature of the various substituents, the molecule can have additional asymmetric centers. Each such asymmetric center will independently produce two optical isomers. All of the possible optical isomers, enantiomers and diastereomers, in mixtures and as pure or partially purified compounds, belong to this invention. The present invention comprehends all such isomeric forms of these compounds. Formula (I) shows the structure of the class of compounds without preferred stereochemistry. The independent syntheses of these optical isomers, or their chromatographic separations, may be achieved as known in the art, appropriately modifying the methodology disclosed therein. Their absolute stereochemistry may be determined by the X-ray crystallography of crystalline products or crystalline intermediates, which are derivatized, if necessary, with a reagent containing an asymmetric center of known absolute configuration. Racemic mixtures of the compounds can be separated into the individual enantiomers by well-known methods, such as coupling of a racemic mixture of compounds to an enantiomerically pure compound to form a diastereomeric mixture, followed by separation of the individual diastereomers by standard methods, such as fractional crystallization or chromatography. The coupling often consists of the formation of salts using an enantiomerically pure acid or base, for example (−)-di-p-toluoyl-D-tartaric acid or (+)-di-p-toluoyl-L-tartaric acid. The diasteromeric derivatives may then be converted to the pure enantiomers by cleavage of the added chiral residue. The racemic mixture of the compounds can also be separated directly by well-known chromatographic methods utilizing chiral stationary phases. Alternatively, any enantiomer of a compound may be obtained by stereoselective synthesis using optically pure starting materials or reagents of known configuration by methods well-known in the art.

Cis and trans isomers of the compound of formula (I), or a pharmaceutically acceptable salt thereof, also belong to the invention, and this also applies to tautomers of the compounds of formula (I).

Some of the crystalline forms for the compounds may exist as polymorphs: as such intended to belong to the invention. Compounds of formula (I) isotopically-labeled to be detectable by PET or SPECT, also fall within the scope of the invention. The same applies to compounds of formula (I) labeled with [¹³C]-, [¹⁴C]-, [³H]-, [¹⁸F]-, [¹²⁵I]- or other isotopically enriched atoms, suitable for receptor binding or metabolism studies.

The compounds of the invention may also be used as reagents or standards in the biochemical study of neurological function, dysfunction and disease.

### DEFINITIONS

General terms used in the description of compounds herein disclosed bear their usual meanings. The term alkyl as used herein denotes a univalent saturated, branched or straight, hydrocarbon chain. The carbon content of various hydrocarbon containing moieties is indicated by a prefix designating the minimum and maximum number of carbon atoms in the moiety, i.e., the prefix C_{x-y} defines the number of carbon atoms present from the integer “x” to the integer “y” inclusive. ‘Alkyl(C₁₋₃)’ for example, includes methyl, ethyl, n-propyl or isopropyl.

To provide a more concise description, the terms ‘compound’ or ‘compounds’ include tautomers, stereoisomers, N-oxides, isotopically-labelled analogues, or pharmacologically acceptable salts, also when not explicitly mentioned.

N-oxides of the compounds mentioned above belong to the invention. Tertiary amines may or may not give rise to N-oxide metabolites. The extent to what N-oxidation takes place varies from trace amounts to a near quantitative conversion. N-oxides may be more active than their corresponding tertiary amines, or less active. Whilst N-oxides can easily be reduced to their corresponding tertiary amines by chemical means, in the human body this happens to varying degrees. Some N-oxides undergo nearly quantitative reductive conversion to the corresponding tertiary amines, in other cases conversion is a mere trace reaction, or even completely absent (Bickel, 1969).

The term ‘form’ encompasses all solids: polymorphs, solvates, and amorphous forms. ‘Crystal form’ refers to various solid forms of the same compound, for example polymorphs, solvates and amorphous forms. ‘Cocrystals’ are multicomponent crystals with a unique lattice: new chemical species produced with neutral compounds. ‘Amorphous forms’ are non-crystalline materials with no long range order, and generally do not give a distinctive powder X-ray diffraction pattern. Crystal forms in general have been described by Byrn (1995) and Martin (1995). ‘Polymorphs’ are crystal structures wherein a compound can crystallize in different crystal packing arrangements, all having the same elemental composition. Polymorphism is a frequently occurring phenomenon, affected by several crystallization conditions such as temperature, level of supersaturation, presence of impurities, polarity of solvent, rate of cooling. Different polymorphs usually have different X-ray diffraction patterns, solid state NMR spectra, infrared or Raman spectra, melting points, density, hardness, crystal shape, optical and electrical properties, stability, and solubility. Recrystallization solvent, rate of crystallization, storage temperature, and other factors may cause one crystal form to dominate.

To give a more concise description, some of the quantitative expressions given herein are not qualified with either “about” or “approximately”. It is understood that whether either of these terms is used explicitly or not, every quantity given is meant to refer to the actual value, and also to the approximation to such given value that would reasonably be inferred based on ordinary skill, including approximations due to experimental or measurement conditions for such given value.

Throughout the description and the claims of this specification the word “comprise” and variations of the word, such as “comprising” and “comprises” is not intended to exclude other additives, components, integers or steps.

While it may be possible for the compounds of formula (I) to be administered as the raw chemical, it is preferable to present them as a ‘pharmaceutical composition’. According to a further aspect, the present invention provides a pharmaceutical composition comprising at least one compound of formula (I), at least one pharmaceutically acceptable salt thereof, or a mixture of any of the foregoing, together with one or more pharmaceutically acceptable carriers thereof, and with or without one or more other therapeutic ingredients. The carrier(s) must be ‘acceptable’ in the sense of being compatible with the other ingredients of the formulation and not deleterious to the recipient thereof. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The affinity of the compounds of the invention for cannabinoid CB₁ receptors was determined as described below. From the binding affinity measured for a given compound of formula (I), one can estimate a theoretical lowest effective dose. At a concentration of the compound equal to twice the measured Kᵢ-value, nearly 100% of the cannabinoid CB₁ receptors likely will be occupied by the compound. By converting that concentration to mg of compound per kg of patient—assuming ideal bioavailability—a theoretical lowest effective dose is obtained. Pharmacokinetic, pharmacodynamic, and other considerations may alter the dose actually administered to a higher or lower value. The dose of the compound to be administered will depend on the relevant indication, the age, weight and sex of the patient and may be determined by a physician. The dosage will preferably be in the range of from 0.01 mg/kg to 10 mg/kg. The typical daily dose of the active ingredients varies within a wide range and will depend on various factors such as the relevant indication, the route of administration, the age, weight and sex of the patient and may be determined by a physician. In general, total daily dose administration to a patient in single or individual doses, may be in amounts, for example, from 0.001 to 10 mg/kg body weight daily, and more usually from 0.01 to 1,000 mg per day, of total active ingredients. Such dosages will be administered to a patient in need of treatment from one to three times each day, or as often as needed for efficacy, and for periods of at least two months, more typically for at least six months, or chronically.

The term “therapeutically effective amount” as used herein refers to an amount of a therapeutic agent to treat a condition treatable by administrating a composition of the invention. That amount includes the amount sufficient to exhibit a detectable therapeutic or ameliorative response in a tissue system, animal or human. The effect may include, for example, treating the conditions listed herein. The precise pharmaceutically effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration.

Thus, it is not useful to specify an exact pharmaceutically effective amount in advance. A “pharmaceutical salt’ refers to an acid:base complex containing an active pharmaceutical ingredient (API) along with additional non-toxic molecular species in the same crystal structure. The term “pharmaceutically acceptable salt” refers to those salts that are, within the scope of sound medical judgment, suitable for use in contact with the tissues of humans and lower animals without undue toxicity, irritation, allergic response, etc., and are commensurate with a reasonable benefit/risk ratio. Pharmaceutically acceptable salts are well-known. They can be prepared in situ when finally isolating and purifying the compounds of the invention, or separately by reacting them with pharmaceutically acceptable non-toxic bases or acids, including inorganic or organic bases and inorganic or organic acids (Berge, 1977). Common anions used in pharmaceutically acceptable salts include: chloride, bromide, sulfate, nitrate, phosphate, bicarbonate, mesylate, esylate, isothianate, tosylate, napsylate, besylate, acetate, propionate, maleate, benzoate, salicylate, fumarate, citrate, lactate, maleate, tartrate, pamoate, succinate, glycolate, hexanoate, octanoate, decanoate, stearate, oleate, aspartate and glutamate. Common cations used as counterions in pharmaceutically acceptable salts include: sodium, potassium, calcium, magnesium, lithium, zinc, aluminum, arginine, lysine, histidine, triethylamine, ethanolamine, triethanolamine, ethilenediamine, meglumine, procaine and benzathine.

The ‘free base’ form may be regenerated by contacting the salt with a base or acid, and isolating the parent compound in the conventional matter. The parent form of the compound differs from the various salt forms in certain physical properties, such as solubility in polar solvents, but otherwise the salts are equivalent to the parent form of the compound for the purposes of the present invention.

The term “treatment” refers to any treatment of a human condition or disease, and includes: (1) inhibiting the disease or condition, i.e., arresting its development, (2) relieving the disease or condition, i.e., causing the condition to regress, or (3) stopping the symptoms of the disease. The term ‘inhibit’ includes its generally accepted meanings: restraining, alleviating, ameliorating, and slowing, stopping or reversing progression, severity, or a resultant symptom. As used herein, the term “medical therapy” intendeds to include diagnostic and therapeutic regimens carried out in vivo or ex vivo on humans.

As used herein ‘obesity’ refers to a condition whereby a person has a Body Mass Index (BMI), calculated as weight per height squared (km/m²), of at least 25.9. Conventionally, those persons with normal weight have a BMI of 19.9 to less than 25.9. The obesity herein may be due to any cause, whether genetic of environmental. Examples of disorders that may result in obesity or be the cause of obesity include overeating and bulimia, polycystic ovarian disease, craniopharyngioma, the Prader-Willi syndrome, Frohlich's syndrome, Type-II diabetes, GH-deficient subjects, normal variant short stature, Turners syndrome, and other pathological conditions showing reduced metabolic activity or a decrease in resting energy expenditure as a percentage of total fat-free mass, e.g. children with acute lymphoblastic leukemia.

### EXAMPLE 1

### General Aspects of Syntheses

The synthesis of a compound of general formula (I), wherein n, R₁, R₂ and R₃ have the meanings as given above, is given in Scheme 1.

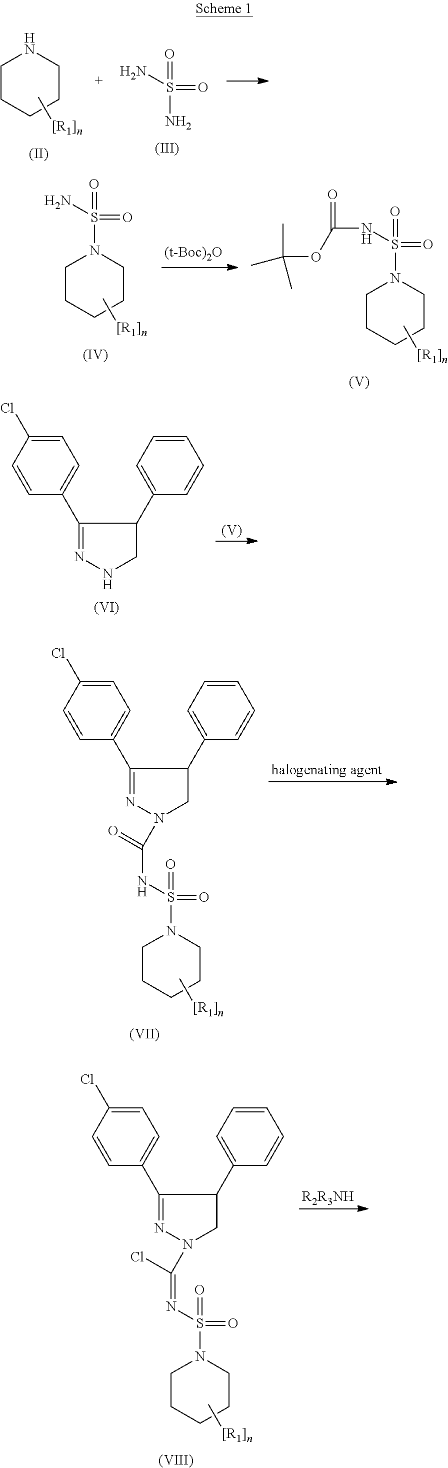

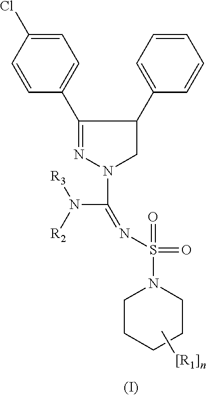

A fluorinated piperidine analog of general formula (II) can be reacted with sulfamide (III) in an inert organic solvent such as butylacetate to give a fluorinated piperidinylsulfonamide of general formula (IV), that can be reacted with tert-Boc anhydride (di-tert-butyl dicarbonate) in an inert organic solvent, such as toluene, in the presence of a base such as triethylamine, and preferably a catalytic amount of 4-dimethylaminopyridine (DMAP) to give a compound of formula (V). The obtained compound of formula (V) can be reacted with 3-(4-chlorophenyl)-4-phenyl-4,5-dihydro-1H-pyrazole (VI) in an inert organic solvent such as toluene, to give a compound of formula (VII). A compound of formula (VII) can be reacted with a halogenating agent, e.g. a chlorinating agent such as POCl₃, in an inert organic solvent such as dichloromethane to give a compound of formula (VIII). Such a reaction is preferably carried out in the presence of DMAP. Compound (VIII) can be reacted with an amine of general formula R₂R₃NH in an inert organic solvent such as dichloromethane to give a compound of general formula (I), wherein n, R₁, R₂ and R₃ have the meanings as given above. The synthesis of a compound of general formula (I), wherein n, R₁, R₂ and R₃ have the meanings as given above and the C₄ atom of the 4,5-dihydro-1H-pyrazole ring has the absolute S configuration is given in Scheme 2.

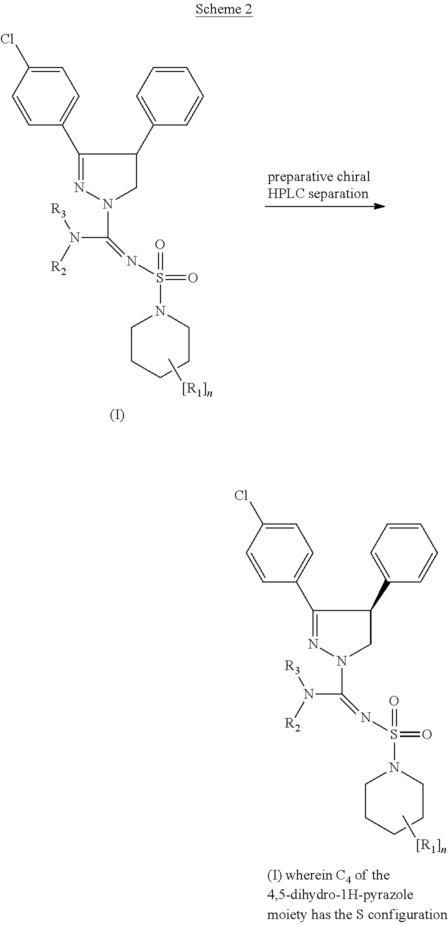

Racemic compound (I) can be separated via chiral preparative HPLC to give compound (I), wherein n, R₁, R₂ and R₃ have the meaning as given above and wherein C₄ of its 4,5-dihydropyrazole moiety has the S configuration.

Compounds described below were prepared according to these procedures. They are intended to further illustrate the invention in more detail, not to restrict the scope of the invention in any way. Other embodiments of the invention will be apparent to skilled persons from consideration of the specification and practice of the invention disclosed herein. The specification and examples must be considered as exemplary only.

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Pharmaceutically acceptable salts may be obtained using well-known procedures, for example by mixing a compound of the present invention with a suitable acid, for instance an inorganic acid or an organic acid.

An X-Ray diffraction analysis can be carried out for crystals of an optically pure compound of the present invention. From the obtained X-ray diffraction data the absolute configuration of the C₄ atom of the pyrazoline ring in an optically pure compound of the present invention can be determined.

### EXAMPLE 2

### Syntheses of Specific Compounds

To be able to distinguish the pharmacological properties of the compounds of the invention from those of related compounds known in the art, reference compounds 21, 24 and 25 (Lange, 2005^{A}) were synthesized as described in WO 03/026648. Compounds 24 and 25 were both obtained in the chiral preparative HLPC step (WO 03/026648, example 100 therein).

### N-[(4,4-difluoropiperidin-1-yl)sulfonyl]-N′-methyl-3-(4-chlorophenyl)-4-phenyl-4,5-dihydro-(1H)-pyrazole-1-carboxamidine (Compound 1)

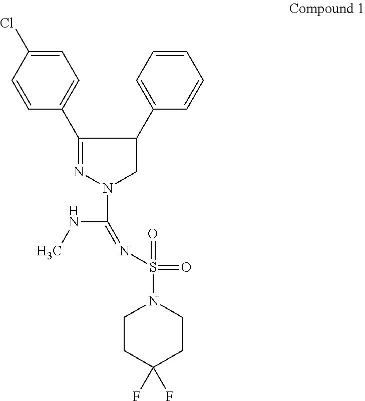

Step 1: Sulfamide (9.15 g; 95.2 mmol) was added to 4,4-difluoropiperidine hydrochloride (15.0 g; 95.2 mmol) in butyl acetate (200 ml). N,N-diisopropylethylamine (DIPEA) (17.9 ml; 104.7 mmol) was added and the magnetically stirred reaction mixture was heated at reflux temperature overnight. The reaction mixture was allowed to attain room temperature. Volatiles were removed in vacuo. Ethyl acetate and 1N HCl were successively added. The organic layer was separated and dried over Na₂SO₄. After filtration, the filtrate was collected and volatiles were removed in vacuo. The product was washed twice with diisopropyl ether affording 4,4-difluoropiperidin-1ylsulfonamide (15.96 g; 83.8%) (Intermediate (IV-1). Melting point: 111-112° C. (recorded on a Büchi 8-545 melting point apparatus, as were all melting points disclosed here). ¹H-NMR (400 MHz, DMSO-d₆): δ 2.02-2.14 (m, 4H); 3.10-3.16 (m, 4H), 6.90 (br s, 2H).

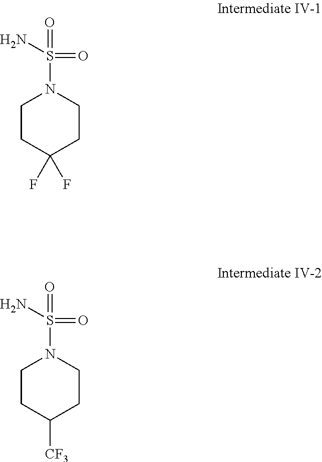

Analogously was prepared: 4-(trifluoromethyl)piperidin-1ylsulfonamide (Intermediate IV-2). Melting point: 160-161° C. ¹H-NMR (400 MHz, DMSO-d₆): δ 1.42-1.55 (m, 2H), 1.86-1.94 (m, 2H), 2.38-2.60 (m, 3H), 3.53-3.60 (m, 2H), 6.63 (br s, 2H). All ¹H NMR spectra disclosed herein, were recorded on a Bruker 400 MHz or 600 MHz instrument using CDCl₃ or DMSO-d₆ as solvent with tetramethylsilane as an internal standard. Chemical shifts are given in ppm (δ scale) downfield from tetramethylsilane. Coupling constants (J) are expressed in Hz.

Step 2: To a magnetically stirred solution of 4,4-difluoropiperidin-1ylsulfonamide (6.0 gram, 30 mmol) was added successively triethylamine (4.4 ml, 31.5 mmol) and DMAP (0.37 g, 3 mmol) and the resulting mixture was heated at 50° C. Di-tert-butyl dicarbonate (7.9 g, 36 mmol) was dropwise added and the resulting mixture was heated at 50° C. for 2 hours. The mixture was allowed to attain room temperature and toluene (100 ml) and hydrochloric acid (50 ml, 1N) were successively added. Layers were separated. The organic layer was successively twice washed with water, dried over Na₂SO₄, filtered and concentrated in vacuo affording N-[(4,4-difluoropiperidin-1-yl)sulfonyl]carbamic acid tert-butyl ester (8.21 g, 91%) (Intermediate (V-1). Melting point: 82-83° C. ¹H-NMR (400 MHz, CDCl₃): δ 1.49 (s, 9H), 2.03-2.15 (m, 4H), 3.53-3.58 (m, 4H), 6.98 (br s, 1H).

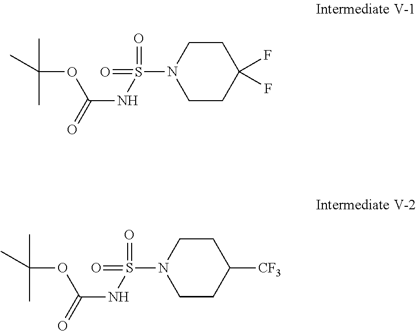

Analogously was prepared: N-[(4-(Trifluoromethyl)piperidin-1-yl)sulfonyl]carbamic acid tert-butyl ester (Intermediate (V-2). Melting point: 104-105° C. ¹H-NMR (400 MHz, CDCl₃): δ 1.50 (s, 9H), 1.63-1.75 (m, 2H), 1.90-1.99 (m, 2H), 2.09-2.25 (m, 1H), 2.90-2.99 (m, 2H), 3.95-4.03 (m, 2H), 6.99 (br s, 1H).

Step 3: To a magnetically stirred solution of N-[(4,4-difluoropiperidin-1-yl)sulfonyl]carbamic acid tert-butyl ester (8.17 g; 27.2 mmol) in toluene was added 3-(4-chlorophenyl)-4-phenyl-4,5-dihydro-1H-pyrazole (7.2 gram, 28 mmol) and the resulting solution was heated at reflux temperature for 3 hours. The mixture was successively allowed to attain room temperature and cooled in an ice-bath. The formed precipitate was collected by filtration, washed twice with toluene and twice with diisopropyl ether to give 3-(4-chlorophenyl)-N-[(4,4-difluoropiperidin-1-yl)sulfonyl]-4-phenyl-4,5-dihydro-1H-pyrazole carboxamide (10 gram, 76% yield) (Intermediate VII-1). Melting point: 215-216° C. ¹H-NMR (400 MHz, CDCl₃): δ 2.06-2.19 (m, 4H), 3.62-3.67 (m, 4H), 3.97 (dd, J=11 and ˜5.5, 1H), 4.39 (t, J=11, 1H), 4.76 (dd, J=11 and ˜5.5 Hz, 1H), 7.15 (br d, J=8 Hz, 2H), 7.25-7.36 (m, 5H), 7.53 (br d, J=8 Hz, 2H), 8.51 (br s, 1H).

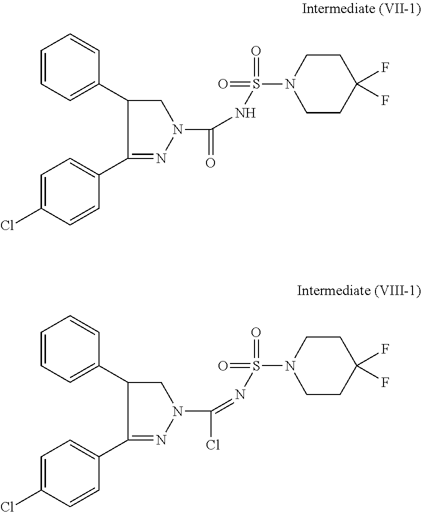

Analogously was prepared: 3-(4-chlorophenyl)-N-[(4-(trifluoromethyl)piperidin-1-yl)sulfonyl]-4-phenyl-4,5-dihydro-1H-pyrazole carboxamide (Intermediate VII-2). Melting point: 112-113° C. ¹H-NMR (400 MHz, CDCl₃): δ 1.67-1.80 (m, 2H), 1.94-2.01 (m, 2H), 2.09-2.23 (m, 1H), 2.98-3.11 (m, 2H), 3.97 (dd, J=12 and ˜5.5, 1H), 4.01-4.10 (m, 2H), 4.38 (t, J=12, 1H), 4.75 (dd, J=12 and ˜5.5 Hz, 1H), 7.13-7.37 (m, 7H), 7.53 (br d, J=8 Hz, 2H), 8.49 (br s, 1H).

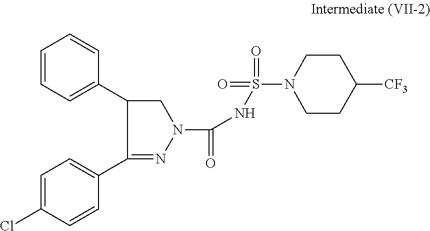

Step 4: To a magnetically stirred solution of 3-(4-chlorophenyl)-N-[(4,4-difluoropiperidin-1-yl)sulfonyl]-4-phenyl-4,5-dihydro-1H-pyrazole carboxamide (10 gram, 20.7 mmol) dissolved in dichloromethane (200 ml) was successively slowly added DMAP (11.2 g; 91.5 mmol), POCl₃ (phosphorus oxychloride) (2.4 ml; 25.6 mmol) in dichloromethane (20 ml). The reaction mixture was heated at reflux temperature for 4 hours. After cooling to 6° C., methylamine hydrochloride (6.3 g; 93.3 mmol) was added to the in situ formed 3-(4-chlorophenyl)-N-[(4,4-difluoropiperidin-1-yl)sulfonyl]-4-phenyl-4,5-dihydro-1H-pyrazole-1-carboximidoyl chloride (Intermediate VIII-1), followed by dropwise addition of DIPEA (23.8 ml; 136.5 mmol). The reaction mixture was stirred overnight at room temperature. Water (120 ml) was added. The layers were separated. The organic layer was successively washed with 1N hydrochloric acid (three times), water (three times), dried over Na₂SO₄, filtered and concentrated in vacuo. Flash chromatographic purification (silica gel, eluant: ethyl acetate) followed by crystallization gave N-[(4,4-difluoropiperidin-1-yl)sulfonyl]-N′-methyl-3-(4-chlorophenyl)-4-phenyl-4,5-dihydro-(1H)-pyrazole-1-carboxamidine (9.32 g; 91% yield) (compound 1). Melting point: 158.5-159.5° C. ¹H-NMR (600 MHz, CDCl₃): δ 2.05-2.14 (m, 4H), 3.24 (d, J=7, 3H), 3.26-3.34 (m, 4H), 4.10-4.18 (m, 1H), 4.57 (t, J=12 Hz, 1H), 4.67 (dd, J=12 and ˜5.5 Hz, 1H), 6.80 (br s, 1H), 7.15 (d, J=8 Hz, 2H), 7.25-7.29 (m, 3H), 7.30-7.35 (m, 2H), 7.53 (d, J=8 Hz, 2H).

Analogously were prepared:

N-[(4-(Trifluoromethyl)piperidin-1-yl)sulfonyl]-N′-methyl-3-(4-chlorophenyl)-4-phenyl-4,5-dihydro-(1H)-pyrazole-1-carboxamidine (compound 2). Melting point: 195-196° C. ¹H-NMR (600 MHz, CDCl₃): δ 1.68-1.78 (m, 2H), 1.88-1.94 (m, 2H), 2.01-2.11 (m, 1H), 2.52-2.61 (m, 2H), 3.25 (d, J=7, 3H), 3.78-3.86 (m, 2H), 4.10-4.17 (m, 1H), 4.57 (t, J=11 Hz, 1H), 4.67 (dd, J=11 and ˜5.5 Hz, 1H), 6.81 (br s, 1H), 7.15 (d, J=8 Hz, 2H), 7.23-7.29 (m, 3H), 7.31-7.34 (m, 2H), 7.52 (d, J=8 Hz, 2H).

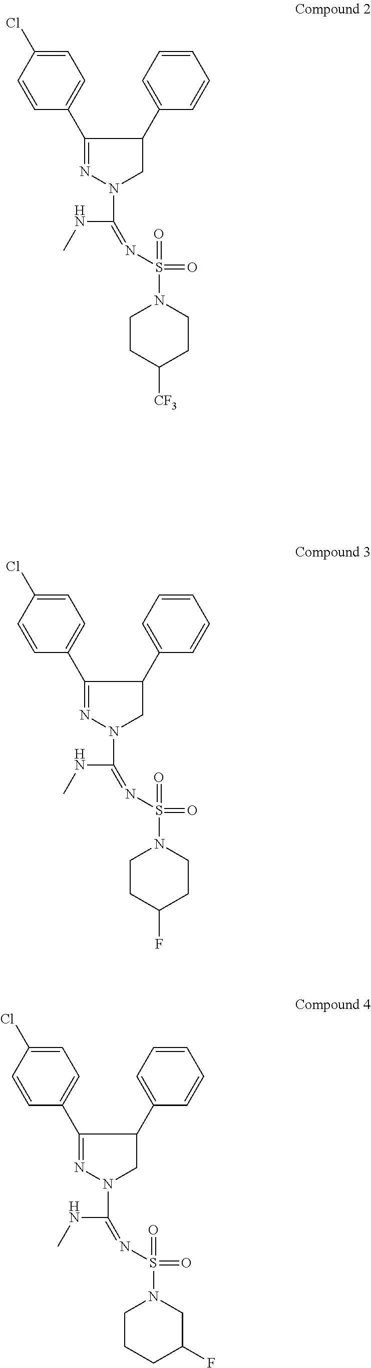

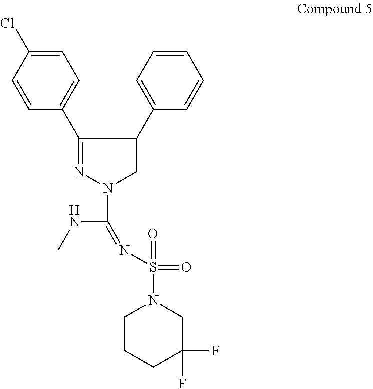

N-[(4-Fluoropiperidin-1-yl)sulfonyl]-N′-methyl-3-(4-chlorophenyl)-4-phenyl-4,5-dihydro-(1H)-pyrazole-1-carboxamidine (compound 3). Melting point: 172-173° C. ¹H-NMR (600 MHz, CDCl₃): δ 1.88-2.00 (m, 4H), 3.11-3.18 (m, 2H), 3.24 (d, J=7, 3H), 3.26-3.33 (m, 2H), 4.11-4.19 (m, 1H), 4.57 (t, J=11 Hz, 1H), 4.66 (dd, J=11 and ˜5.5 Hz, 1H), 4.71-4.75 and 4.79-4.83 (m, 1H), 6.81 (br s, 1H), 7.15 (d, J=8 Hz, 2H), 7.23-7.28 (m, 3H), 7.30-7.34 (m, 2H), 7.52 (d, J=8 Hz, 2H).

N-[(3-Fluoropiperidin-1-yl)sulfonyl]-N′-methyl-3-(4-chlorophenyl)-4-phenyl-4,5-dihydro-(1H)-pyrazole-1-carboxamidine (compound 4). Melting point: 170-171° C. ¹H-NMR (400 MHz, CDCl₃): δ 1.60-1.72 (m, 2H), 1.84-1.98 (m, 2H), 2.91-3.09 (m, 2H), 3.20-3.30 (m, 4H), 3.46-3.57 (m, 1H), 4.10-4.18 (m, 1H), 4.53-4.82 (m, 3H), 6.86 (br s, 1H), 7.15 (d, J=8 Hz, 2H), 7.23-7.36 (m, 5H), 7.53 (d, J=8 Hz, 2H).

N-[(3,3-Difluoropiperidin-1-yl)sulfonyl]-N′-methyl-3-(4-chlorophenyl)-4-phenyl-4,5-dihydro-(1H)-pyrazole-1-carboxamidine (compound 5). Melting point: 166-167° C. ¹H-NMR (400 MHz, CDCl₃): δ 1.81-1.99 (m, 4H), 3.10-3.20 (m, 2H), 3.25 (d, J=7, 3H), 3.29-3.38 (m, 2H), 4.15 (dd, J=11 and 5 Hz, 1H), 4.57 (t, J=11 Hz, 1H), 4.67 (dd, J=11 and 5 Hz, 1H), 6.83 (br s, 1H), 7.16 (d, J=8 Hz, 2H), 7.23-7.36 (m, 5H), 7.53 (d, J=8 Hz, 2H).

N-[(4,4-Difluoropiperidin-1-yl)sulfonyl]-N′-ethyl-3-(4-chlorophenyl)-4-phenyl-4,5-dihydro-(1H)-pyrazole-1-carboxamidine (compound 6). ¹H-NMR (400 MHz, CDCl₃): δ 1.33 (t, J=7, 3H), 2.02-2.15 (m, 4H), 3.25-3.33 (m, 4H), 3.66-3.76 (m, 2H), 4.10-4.18 (m, 1H), 4.51-4.70 (m, 2H), 6.73 (br s, 1H), 7.15 (br d, J=8 Hz, 2H), 7.23-7.37 (m, 5H), 7.52 (br d, J=8 Hz, 2H).

N²-[(4,4-Difluoropiperidin-1-yl)sulfonyl]-N¹-dimethyl-3-(4-chlorophenyl)-4-phenyl-4,5-dihydro-(1H)-pyrazole-1-carboxamidine (compound 7). ¹H-NMR (400 MHz, CDCl₃): δ 2.02-2.15 (m, 4H), 3.23 (s, 6H), 3.25-3.33 (m, 4H), 3.93-3.97 (m, 1H), 4.53-4.65 (m, 2H), 7.18 (br d, J=8 Hz, 2H), 7.23-7.37 (m, 5H), 7.55 (br d, J=8 Hz, 2H).

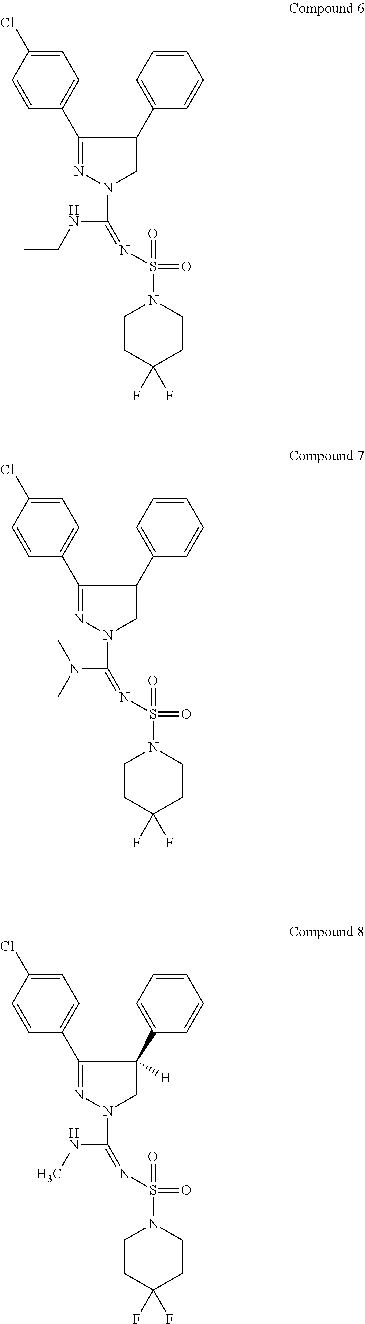

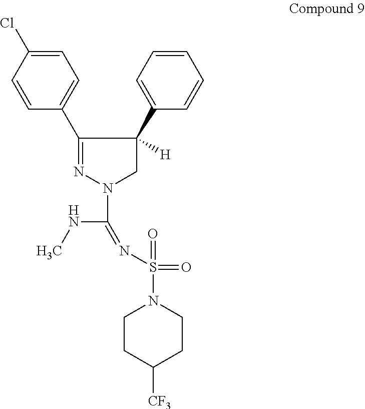

(−)-(4S)—N-[(4,4-difluoropiperidin-1-yl)sulfonyl]-N′-methyl-3-(4-chlorophenyl)-4-phenyl-4,5-dihydro-(1H)-pyrazole-1-carboxamidine, compound 8 (3.74 gram), was obtained via preparative chiral chromatographic separation of 8.2 gram racemic N-[(4,4-difluoropiperidin-1-yl)sulfonyl]-N′-methyl-3-(4-chlorophenyl)-4-phenyl-4,5-dihydro-(1H)-pyrazole-1-carboxamidine (compound 1). Retention time, preparative column: 7.5 minutes. Compound 8: Melting point: 185.5-186° C. [α²⁵_{D}]=−148°, c=1, methanol (Optical rotations were measured on a Bellingham/Stanley ADP410 polarimeter. Specific rotations ([α²⁵_{D}]) are given as deg/dm, the concentration values are reported as g/100 ml of the specified solvent). ¹H-NMR (400 MHz, CDCl₃): δ 2.03-2.16 (m, 4H), 3.24 (d, J=7, 3H), 3.26-3.34 (m, 4H), 4.14 (dd, J=12 and ˜5.5 Hz, 1H), 4.57 (t, J=12 Hz, 1H), 4.67 (dd, J=12 and ˜5.5 Hz, 1H), 6.79 (br s, 1H), 7.15 (d, J=8 Hz, 2H), 7.23-7.35 (m, 5H), 7.53 (d, J=8 Hz, 2H). Enantiomeric excess: >99%.

Preparative chiral HPLC method: A 250×80 mm column was used. Stationary phase: CHIRALPAK® AD 20 μm. Methanol/acetonitrile=90/10 (v/v) was used as the mobile phase. Flow rate: 200 ml/minute. Temperature: room temperature. Detection UV 230 nm

Analytical HPLC monitoring system: A 250×4.6 mm column was used. Stationary phase: CHIRALPAK® AD 10 μm. Methanol/acetonitrile=90/10 (v/v)+0.1% diethylamine was used as mobile phase. Flow rate: 1 ml/minute. Temperature: room temperature. Detection UV 230 nm.

(−)-(4S)—N-[(4-(Trifluoromethyl)piperidin-1-yl)sulfonyl]-N′-methyl-3-(4-chlorophenyl)-4-phenyl-4,5-dihydro-(1H)-pyrazole-1-carboxamidine, compound 9 (4.16 gram), was obtained via preparative chiral chromatographic separation of 8.55 gram racemic N-[(4-(trifluoromethyl)piperidin-1-yl)sulfonyl]-N′-methyl-3-(4-chlorophenyl)-4-phenyl-4,5-dihydro-(1H)-pyrazole-1-carboxamidine (compound 2). Retention time, preparative column: 3.5 minutes. Compound 9: [α²⁵_{D}]=−130°, c=1, methanol. ¹H-NMR (400 MHz, CDCl₃): δ 1.67-1.80 (m, 2H), 1.88-1.95 (m, 2H), 1.99-2.13 (m, 1H), 2.51-2.63 (m, 2H), 3.25 (d, J=7, 3H), 3.77-3.86 (m, 2H), 4.10-4.17 (m, 1H), 4.57 (t, J=11 Hz, 1H), 4.67 (dd, J=11 and ˜5.5 Hz, 1H), 6.81 (br s, 1H), 7.15 (d, J=8 Hz, 2H), 7.23-7.36 (m, 5H), 7.52 (br d, J=8 Hz, 2H). Enantiomeric excess: >99. Melting point: 164.5-165° C.

(+)-(4R)—N-[(4-(Trifluoromethyl)piperidin-1-yl)sulfonyl]-N′-methyl-3-(4-chlorophenyl)-4-phenyl-4,5-dihydro-(1H)-pyrazole-1-carboxamidine, compound 10 (22.3 g), was obtained by preparative chiral chromatographic separation of 50 gram racemic N-[(4-(trifluoromethyl)piperidin-1-yl)sulfonyl]-N′-methyl-3-(4-chlorophenyl)-4-phenyl-4,5-dihydro-(1H)-pyrazole-1-carboxamidine (compound 2). Compound 10: [α²⁵_{D}]=+126°, c=1, methanol. Enantiomeric excess: >99. NMR spectrum and melting point were the same as those of compound 9.

Preparative chiral HPLC method: A 250×76 mm column was used. Stationary phase: CHIRALPAK® AD 20 μm. Methanol/acetonitrile=50/50 (v/v) was used as the mobile phase. Flow rate: 270 ml/minute. Temperature: 25° C. Detection UV 250 nm

Analytical HPLC monitoring system: A 250×4.6 mm column was used. Stationary phase: CHIRALPAK® AD-H 5 μm. Methanol/acetonitrile=50/50 (v/v) was used as the mobile phase. Flow rate: 1 ml/minute. Temperature: room temperature. Detection: Diode array detection (DAD) 230 nm.

### EXAMPLE 3

### Pharmacological Methods

In vitro affinity for cannabinoid-CB₁ receptors was determined using membrane preparations of Chinese hamster ovary (CHO) cells wherein human cannabinoid CB₁ receptors were stably transfected in conjunction with [³H]CP-55,940 as radioligand. After incubation of a freshly prepared cell membrane preparation with the [³H]-ligand, with or without addition of compounds of the invention, separation of bound and free ligand was performed by filtration over glassfiber filters. Radioactivity on the filter was measured by liquid scintillation counting. The binding data were either obtained by CEREP (128, rue Danton, 92500 Rueil-Malmaison, France) or at Solvay Pharmaceuticals B.V. (C. J. van Houtenlaan 36, 1381 CP Weesp, The Netherlands).

In vitro affinity for cannabinoid-CB₂ receptors was determined using membrane preparations of CHO cells wherein human cannabinoid CB₂ receptors were stably transfected in conjunction with [³H]CP-55,940 as radioligand. After incubation of a freshly prepared cell membrane preparation with the [³H]-ligand, with or without addition of compounds of the invention, separation of bound and free ligand was performed by filtration over glassfiber filters. Radioactivity on the filter was measured by liquid scintillation counting.

In vivo cannabinoid-CB₁ receptor antagonism was assessed with the CP-55,940-induced hypotension test in rat. Male normotensive rats (225-300 g; Harlan, Horst, The Netherlands) were anaesthetized with pentobarbital (80 mg/kg ip). Blood pressure was measured, via a cannula inserted into the left carotid artery, by means of a Spectramed DTX-plus pressure transducer (Spectramed B.V., Bilthoven, The Netherlands). After amplification by a Nihon Kohden Carrier Amplifier (Type AP-621G; Nihon Kohden B.V., Amsterdam, The Netherlands), the blood pressure signal was registered on a personal computer (Compaq Deskpro 386s), by means of a Po-Ne-Mah data-acquisition program (Po-Ne-Mah Inc., Storrs, USA). Heart rate was derived from the pulsatile pressure signal. All compounds were administered orally as a microsuspension in 1% methylcellulose 30 minutes before induction of the anesthesia, 60 minutes prior to administration of the CB₁ receptor agonist CP-55,940. The injection volume was 10 ml/kg. After haemodynamic stabilization the CB₁ receptor agonist CP-55,940 (0.1 mg/kg i.v.) was administered and the hypotensive effect established (Wagner 2001).

### EXAMPLE 4

### Pharmacological Test Results

When compared to the racemic compound 21 (Lange, 2005^{A}), the racemic compounds of the invention, compounds 1-7, have considerably higher affinities for human CB₁ receptors: factors vary from 6-50 fold. Compound 24 (Lange, 2005^{A}) is the active (S)-(−)-enantiomer. Also for the compounds of the invention, the active (S)-(−)-enantiomers, compounds 8 and 9 have much higher affinities for human CB₁ receptors than their non-fluorinated analog compound 24 (Lange, 2005^{A}): factors are 12 and 19 respectively.

In addition, compound 9 was found more active in vivo after oral administration in the CB₁ mediated (CP-55,940-induced) hypotension test than the non-fluorinated compound 24 (Lange, 2005^{A}).

To illustrate that the (S)-(−)-enantiomers are the eutomers, and the (R)-(+)-enantiomers the distomers, compound 10 was isolated and tested. It was shown to possess an affinity for human CB₁ receptors about 40-fold less than that of compound 9, and devoid of in vivo activity when given at 30 mg/kg.

Compounds 8 and 9 are highly selective CB₁ receptor ligands, showing more than 100 fold selectivity over CB₂ receptors. Thus, also CB₁/CB₂ selectivities of compounds of the invention are higher than that of the non-fluorinated compound 24 (Lange, 2005^{A}).

In the table below, in vitro and in vivo pharmacological test results, obtained using the protocols given above, are compiled. All data are means from at least two independent experiments.

| | | | | in vivo |
|---|---|---|---|---|
| | | in vitro pharmacology | | pharmacology |
| | | receptor binding | | blood pressure |
| | | h-CB₁ | h-CB₂ | rat CB₁ |
| | | receptor | receptor | receptor |
| | (4)-stereo | Kᵢ (nM) | Kᵢ (nM) | ED₅₀ (mg/kg, p.o.) |
| references * | | | | |
| compound 21 | racemic | 152 | | |
| compound 24 | (S)-(−)- | 58 | 3,495 | 8 |
| compound 25 | (R)-(+)- | 763 | | |
| invention | | | | |
| compound 1 | racemic | 10 | | |
| compound 8 | (S)-(−)- | 5 | 1,000 | |
| compound 2 | racemic | 3 | | |
| compound 9 | (S)-(−)- | 3 | 316 | 2 |
| compound 10 | (R)-(+)- | 125 | >1,000 | >30 |
| compound 3 | racemic | 5 | | |
| compound 4 | racemic | 16 | | |
| compound 5 | racemic | 10 | | |
| compound 6 | racemic | 10 | | |
| compound 7 | racemic | 25 | | |

* references: compounds 21, 24 and 25 as described (Lange, 2005^{A});

### EXAMPLE 5

### Pharmaceutical Preparations

For clinical use, compounds of formula (I) are formulated into pharmaceutical compositions that are novel embodiments of the invention because they contain the compounds, more particularly specific compounds disclosed herein. Types of pharmaceutical compositions that may be used include: tablets, chewable tablets, capsules (including microcapsules), solutions, parenteral solutions, ointments (creams and gels), suppositories, suspensions, and other types disclosed herein, or are apparent to a skilled person from the specification and general knowledge. The active ingredient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains at least one compound of formula (I) in admixture with at least one pharmaceutically acceptable adjuvant, diluent and/or carrier. The total amount of active ingredients suitably can be in the range of from about 0.1% (w/w) to about 95% (w/w) of the formulation, suitably from 0.5% to 50% (w/w) and preferably from 1% to 25% (w/w). In some embodiments, the amount of active ingredient can be greater than about 95% (w/w) or less than about 0.1% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents like sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet can be prepared using the ingredients below:

| Ingredient | Quantity (mg/tablet) |
|---|---|
| COMPOUND No. 9 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and ‘kits of parts’ comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human administration. The use of formulations of the invention in the manufacture of medicaments for use in the treatment of a condition wherein modulation of cannabinoid CB₁ receptors is required or desired, and methods of medical treatment or comprising the administration of a therapeutically effective total amount of at least one compound of formula (I), either as such or, in the case of prodrugs, after administration, to a patient suffering from, or susceptible to, a condition wherein modulation of cannabinoid CB₁ receptors is required or desired.

By way of example and not of limitation, several pharmaceutical compositions are given, comprising active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### BIBLIOGRAPHY

To the extend in which the following references are useful to one skilled in the art, or to more fully describe this invention, they are incorporated herein by reference. Neither these, nor any other documents or quotes cited herein, nor citations to any references, are admitted to be prior art documents or citations.
Antel, J.; Gregory, P. C.; Nordheim, U. J Med Chem 2006, 49, 4008.
Barth, F. Ann Rep Med Chem 2005, 40, 103.
Berge, S. M.: “Pharmaceutical salts”, J. Pharmaceutical Science, 66, 1-19 (1977).
Bickel, M. H.: “The pharmacology and Biochemistry of N-oxides”, Pharmacological Reviews, 21(4), 325-355, 1969.
Byrn et al., Pharmaceutical Research, 12(7), 945-954, 1995.
Castellano, C. et al., Curr Drug Targets CNS Neurol Dis., 2003, 2, 389.
Colombo, G.; Agabio, R.; Diaz, G.; Lobina, C.; Reali, R., Gess, G. L., Life Sci, 1998, 63, 113.
Cooke, D.; Bloom, S, Nat Rev Drug Discov, 2006, 5, 919.
Costa, B. Br J Pharmacol 2007, 150, 535.
Crozi, T.; Zarini, E. Br J Pharmacol 2007, 150, 559.
De Petrocellis, L. et al., Br. J. Pharmacol. 2004, 141, 765.
Di Marzo, V. et al., Nature Rev. Drug Discov. 2004, 3, 771.
Di Marzo, V. Nature Rev. Drug Discov. 2008, 7, 438.
Hepworth, D.; Carpino, P. A.; Black, S. C. Ann Rep Med Chem 2006, 41, 77.
Högenauer, E. K. Expert Opin Ther Patents 2007, 17, 1457.
Lambert, D. M. and Fowler, C. J. J. Med. Chem. 2005, 48, 5059.
Lambert. D. M. Expert Opin Ther Patents 2007, 17, 1027.
Lange, J. H. M.; Kruse, C. G. Curr Opin Drug Discovery Dev 2004, 7, 498.
Lange J. H. M., et al. Bioorg. Med. Chem. Lett. 2005^{A}, 47, 4794-4798.
Lange, J. H. M.; Kruse, C. G. Drug Discov Today 2005^{B}, 10, 693.
Le Foll, B.; Goldberg, S. R. J Pharmacol Exp Ther 2005, 312, 875.
Maldonado, R.; Valverde, O.; Berrendero, F. Trends Neurosci 2006, 29, 225.
Martin, E. W. (Editor), “Remington: The Science and Practice of Pharmacy”, Mack Publishing Company, 19^{th} Edition, Easton, Pa, Vol 2., Chapter 83, 1447-1462, 1995.
Muccioli, G. G.; Lambert, D. M. Expert Opin Ther Patents 2006, 16 1405.
van Gaal, L. F.; Rissanen, A. M.; Scheen, A. J.; Ziegler, O.; Roessner, S. Lancet 2005, 365, 1389.
Sarnataro, D., et al., Mol Pharmacol., 2006, 70, 1298.
Teixeira-Clerc, F., et al., Nature Med, 2006, 12, 671.
Vandevoorde, S, and Lambert, D. M. Curr. Pharm. Des. 2005, 11, 2647.
Wagner, J. A., et al., Hemodynamic effects of cannabinoids: coronary and cerebral vasodilation mediated by cannabinoid CB₁ receptors. Eur. J. Pharmacol. 2001, 423, 203-10).
WO 03/026648
Wolff, M. C.; Leander, J. D. Eur J Pharmacol 2003, 477, 213.

1-12. (canceled)

13. A compound of formula (I): 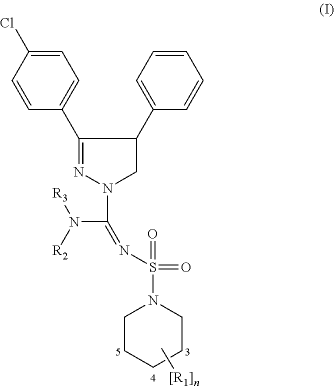
or a tautomer, stereoisomer, or N-oxide thereof, or a pharmacologically acceptable salt of any of the foregoing, wherein:
n is 1 or 2,
when n is 1, R₁ is chosen from F and CF₃, and is bonded to either the carbon atom at the 3-position or the carbon atom at the 4-position of the piperidine ring,
when n is 2, R₁ is F, and one R₁ is bonded to the carbon atom at the 3-position of the piperidine ring and the second R₁ is bonded to either the carbon atom at the 4-position or the carbon atom at the 5-position of the piperidine ring,
R₂ is chosen from H and (C₁₋₃)-alkyl, and
R₃ is chosen from H and methyl.

14. The compound of claim 13, wherein R₃ is H.

15. The compound of claim 13, wherein R₂ is chosen from methyl and ethyl, and R₃ is H.

16. The compound of claim 13, wherein the compound is chosen from:
N-[(4,4-difluoropiperidin-1-yl)sulfonyl]-N′-methyl-3-(4-chlorophenyl)-4-phenyl-4,5-dihydro-(1H)-pyrazole-1-carboxamidine,
N-[(4-(Trifluoromethyl)piperidin-1-yl)sulfonyl]-N′-methyl-3-(4-chlorophenyl)-4-phenyl-4,5-dihydro-(1H)-pyrazole-1-carboxamidine,
N-[(4-Fluoropiperidin-1-yl)sulfonyl]-N′-methyl-3-(4-chlorophenyl)-4-phenyl-4,5-dihydro-(1H)-pyrazole-1-carboxamidine,
N-[(3-Fluoropiperidin-1-yl)sulfonyl]-N′-methyl-3-(4-chlorophenyl)-4-phenyl-4,5-dihydro-(1H)-pyrazole-1-carboxamidine,
N-[(3,3-Difluoropiperidin-1-yl)sulfonyl]-N′-methyl-3-(4-chlorophenyl)-4-phenyl-4,5-dihydro-(1H)-pyrazole-1-carboxamidine,
N-[(4,4-Difluoropiperidin-1-yl)sulfonyl]-N′-ethyl-3-(4-chlorophenyl)-4-phenyl-4,5-dihydro-(1H)-pyrazole-1-carboxamidine,
N²-[(4,4-Difluoropiperidin-1-yl)sulfonyl]-N¹-dimethyl-3-(4-chlorophenyl)-4-phenyl-4,5-dihydro-(1H)-pyrazole-1-carboxamidine,
(−)-(4S)—N-[(4,4-difluoropiperidin-1-yl)sulfonyl]-N′-methyl-3-(4-chlorophenyl)-4-phenyl-4,5-dihydro-(1H)-pyrazole-1-carboxamidine,
(−)-(4S)—N-[(4-(trifluoromethyl)piperidin-1-yl)sulfonyl]-N′-methyl-3-(4-chlorophenyl)-4-phenyl-4,5-dihydro-(1H)-pyrazole-1-carboxamidine, and
(+)-(4R)—N-[(4-(trifluoromethyl)piperidin-1-yl)sulfonyl]-N′-methyl-3-(4-chlorophenyl)-4-phenyl-4,5-dihydro-(1H)-pyrazole-1-carboxamidine.

17. The compound of claim 13, wherein said compound is an optically active enantiomer.

18. The compound of claim 13, wherein the carbon atom at the 4-position of the 4,5-dihydropyrazole ring is the (S)-enantiomer.

19. A compound of formula (VII): 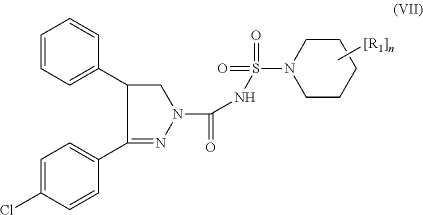
wherein
n is 1 or 2,
when n is 1, R₁ is chosen from F and CF₃, and is bonded to either the carbon atom at the 3-position or the carbon atom at the 4-position of the piperidine ring, and
when n is 2, R₁ is F, and one R₁ is bonded to the carbon atom at the 3-position of the piperidine ring and the second R₁ is bonded to either the carbon atom at the 4-position or the carbon atom at the 5-position of the piperidine ring.

20. A compound of formula (VIII): 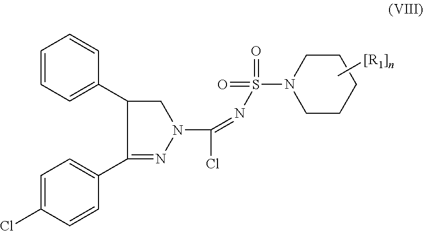
wherein
n is 1 or 2,
when n is 1, R₁ is chosen from F and CF₃, and is bonded to either the carbon atom at the 3-position or the carbon atom at the 4-position of the piperidine ring, and
when n is 2, R₁ is F, and one R₁ is bonded to the carbon atom at the 3-position of the piperidine ring and the second R₁ is bonded to either the carbon atom at the 4-position or the carbon atom at the 5-position of the piperidine ring.

21. A method for preparing a compound of formula (I) according to claim 13, said method comprising:
(i) reacting a fluorinated piperidine analog of formula (II) 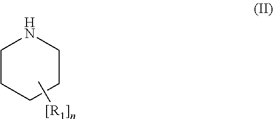
wherein n is 1 or 2,
when n is 1, R₁ is chosen from F and CF₃, and is bonded to either the carbon atom at the 3-position or the carbon atom at the 4-position of the piperidine ring, and
when n is 2, R₁ is F, and one R₁ is bonded to the carbon atom at the 3-position of the piperidine ring and the second R₁ is bonded to either the carbon atom at the 4-position or the carbon atom at the 5-position of the piperidine ring,
with sulfamide (III): 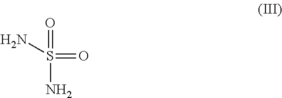
in an inert organic solvent to give a fluorinated piperidinyl-sulfonamide of formula (IV): 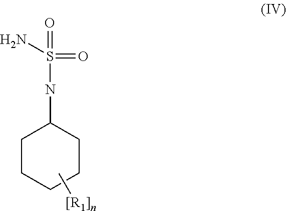
(ii) reacting the fluorinated piperidinylsulfonamide of formula (IV) with tert-Boc anhydride in an inert organic solvent in the presence of a base, to give a compound of formula (V): 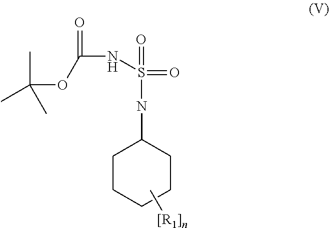
(iii) reacting the compound of formula (V) with 3-(4-chlorophenyl)-4-phenyl-4,5-dihydro-1H-pyrazole (VI) in an inert organic solvent, to give a compound of formula (VII), 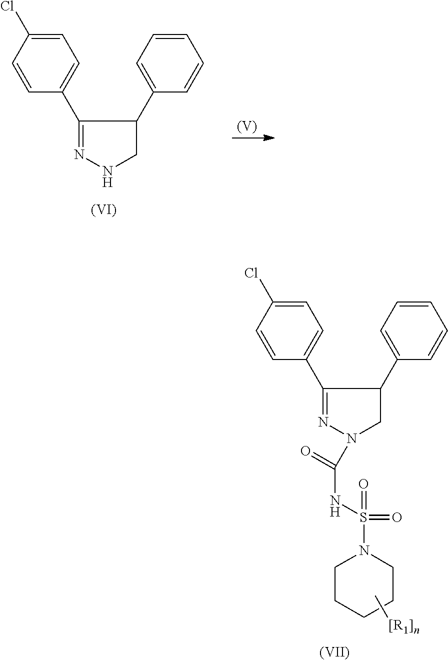
(iv) reacting the compound of formula (VII) with a halogenating agent in an inert organic solvent to give a compound of formula (VIII): 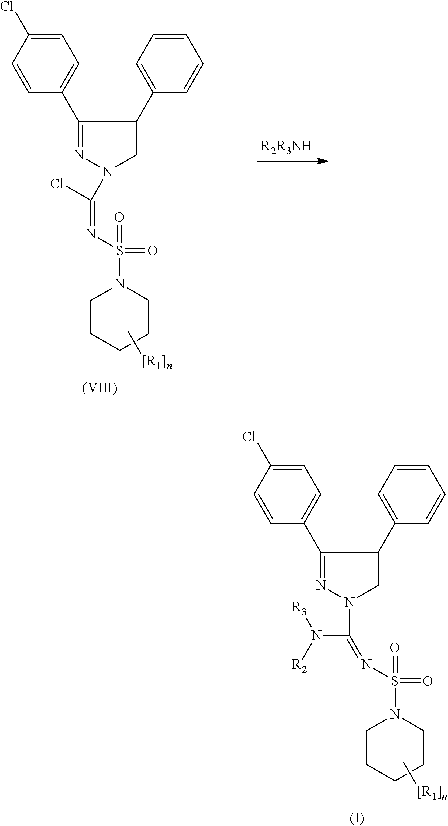
(v) reacting the compound of formula (VIII) with an amine of formula R₂R₃NH, wherein R₂ is chosen from H and (C₁₋₃)-alkyl and R₃ is chosen from H and methyl, in an inert organic solvent, to give a compound of formula (I), and
(vi) purifying the compound of formula (I) by chiral preparative HPLC to give compound (I), wherein C₄ of the 4,5-dihydropyrazole moiety has the S configuration

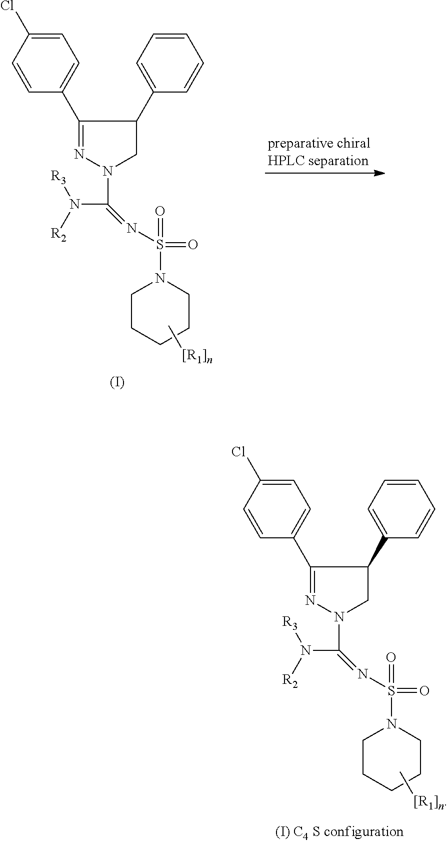

22. The method of claim 21, wherein at least one reaction chosen from (ii) and (iv) further comprises a catalytic amount of 4-dimethyl aminopyridine.

23. The method of claim 21, wherein the inert organic solvent of reaction (i) is butylacetate.

24. The method of claim 21, wherein the inert organic solvent of reaction (ii) is toluene and the base of reaction (ii) is triethylamine.

25. The method of claim 21, wherein the halogenating agent of reaction (iv) is POCl₃ and the inert organic solvent of reaction (iv) is dichloromethane.

26. The method of claim 21, wherein the inert organic solvent of reaction (v) is dichloromethane.

27. A method for treating a condition in a patient in need thereof comprising administering to the patient a therapeutically effective amount of a compound of claim 13, wherein the condition is chosen from obesity and obesity-related cardiovascular disorders, drug addiction, cognition deficits, liver fibrosis, and inflammatory disorders.

28. A pharmaceutical composition comprising:
a pharmacologically active amount of at least one compound of claim 13 or a pharmacologically acceptable salt thereof, and
at least one pharmaceutically acceptable carrier, at least one pharmaceutically acceptable auxiliary substance, or a combination of two or more thereof.
